(19) United States
(12) Patent Application Publication
(10) Pub. No.: US 2008/0128423 A1
(43) Pub. Date: Jun. 5, 2008

(54) Self-warming moisturizing lotion in a dual chamber bottle

(76) Inventors: Donald Rick, Dumont, NJ (US); Ammunje S. Navak, Great Meadows, NJ (US); Gurpreet Bhathal, East Windsor, NJ (US)

Correspondence Address: Evan J. Federman;Warner-Lambert Company LLC, 201 Tabor Road, Morris Plains, NJ, 07950, US

(21) Appl. No.: 11/607,531

(22) Filed: Dec. 1, 2006

(51) Int. Cl. B65D 25/38; A61K 47/02; A61K 47/14; A61K 47/12; A61K 47/10; A61K 47/38; A61Q 99/00

(52) U.S. Cl. 220/500; 514/770; 514/785; 514/784; 514/781

(57) ABSTRACT

The present invention contemplates a dual warming and moisturizing composition comprising a hydrous portion comprising water and an anhydrous portion comprising a zeolite a carrier and a suspending or dispersing agent, wherein the anhydrous portion and the hydrous portion are combined in use to provide warming and moisturizing effects without the addition of external water.

### FIELD OF THE INVENTION

The present invention relates to a composition providing both heating and moisturizing benefits which composition can be in the form of a liquid, cream, lotion, gel or paste. In particular, the present invention relates to a composition comprising an anhydrous heating portion which is adapted to evolve heat when it comes into contact with the hydrous moisturizing portion.

### BACKGROUND

Heat-producing compositions produce a very pleasant sensation. These formulations have better aesthetics than the traditional cold products applied to the skin because of the warming properties of these compositions.

Compositions relating to the generation of heat typically include finely divided solid adsorbent materials which are capable of exothermally reacting with water, such as silica gels, activated alumina, and synthetic zeolites. These compositions typically have a zeolite combined with sodium ions or potassium ions. In addition, there are other types of heating compositions that have other exothermic reagents reactive with water such as kaolin, Fuller's Earth, china clay and bentonite.

A common problem in the art is that the heating composition relies on the water that is not part of the composition to generate the warming effects, limiting their use to locations with available water.

For example, U.S. Pat. No. 6,752,998 to Verdrel-Lahaxe et al., which is incorporated herein by reference in its entirety, discloses an exothermic composition for the cleansing or removing of make-up which contains zeolites as the heating component. The skin is moistened with water prior to use, and then is rinsed off the skin.

Likewise, U.S. Pat. App. Pub. No. 2006/0067957 to Hwang et al. discloses a skin-cleansing composition containing zeolites which is self-warming upon contact with water from an external source. The composition is also rinsed off after cleansing.

Finally, U.S. Pat. App. Pub. No. 2006/0110415 to Gupta discloses two zeolite containing arthritis pain relief creams which provide heat release upon application to pre-wetted skin, and U.S. Pat. No. 7,067,140 to Koike et al. discloses a massage aid composition containing zeolites which provides a calefacient feeling upon contact with pre-wetted skin.

As can be seen, these products require wetting of the face or skin before use of the product. Further, none of these compositions provide for moisturization of the skin.

Accordingly, it would be desirable to provide an improved composition which provides both warming and moisturization without the use of added water from an external source and which is stable until activated.

It is, therefore, an object of the present invention to provide a dual warming and moisturizing composition.

It is another object of the present invention to provide a dual warming and moisturizing composition, as above, which consists of an anhydrous portion and a hydrous portion.

It is yet another object of the present invention to provide a dual warming and moisturizing composition, as above, which is stably stored in a dual chambered container.

It is a further object of the present invention to provide a dual warming and moisturizing composition stored in a dual chambered container, as above, wherein the anhydrous and hydrous portions are dispensed simultaneously.

It is still another object of the present invention to provide a dual warming and moisturizing composition, as above, wherein the anhydrous portion contains a zeolite and a carrier.

It is still a further object of the present invention to provide a dual warming and moisturizing composition stored in a dual chambered container, as above, wherein the anhydrous portion contains a sufficient zeolite concentration to provide a pleasant warming effect upon mixing with the hydrous portion.

It is still a further object of the present invention to provide a dual warming and moisturizing composition stored in a dual chambered container, as above, wherein the anhydrous and hydrous portions are dispensed at a constant rate.

It is a further object of the present invention to provide a dual warming and moisturizing composition, as above, wherein the anhydrous and hydrous portions have a substantially similar viscosity.

It is yet a further object of the invention to provide a dual chambered kit for dispensing a dual warming and moisturizing composition.

These and other objects of the present invention, as well as the advantages thereof over existing prior art relating to skin moisturizing compositions, which will become apparent from the description to follow, are accomplished by the improvements hereinafter described and claimed.

### SUMMARY OF THE INVENTION

Various aspects of the invention are directed to novel warming compositions useful for topical application to human skin. The novel compositions of the present invention are particularly desirable in that they provide self-heating and moisturizing properties without the need of adding water to the composition to achieve heating effects. Specifically, the novel compositions of the present invention are useful for topical application to human skin.

One aspect of the invention relates to a dual warming and moisturizing composition comprising a hydrous portion and an anhydrous portion. The anhydrous portion comprises, for example, a zeolite and a carrier. The hydrous portion comprises water and additional selected ingredients which comprise lotions having a water content of at least 10 weight %. The anhydrous portion and the hydrous portion are combined to provide warming and moisturizing effects in a single composition as they are mixed upon application to the skin.

Another aspect of the invention relates to a product comprising a dual chambered container for dispensing both the anhydrous portion and the hydrous portion at the same time in the ratios described, especially in approximately equal amounts. The dual chambered container comprises a first chamber containing the anhydrous portion and a second chamber containing the hydrous portion.

A preferred embodiment of the present invention provides a dual warming and moisturizing composition which comprises a hydrous portion comprising a moisturizing composition having a water content of at least 10 weight %; and an anhydrous portion comprising a zeolite, a carrier and a suspending or a dispersing agent; wherein (i) the anhydrous portion and the hydrous portion are provided in a single container which keeps said portions separate until use (ii) the concentration of zeolite in the anhydrous portion is matched to the water content of the hydrous portion to provide a pleasant warming effect upon use and (iii) the anhydrous portion and the hydrous portion provide warming and moisturizing effects without externally added water when said portions are mixed together.

Another aspect of the present invention provides that the mixing the anhydrous and hydrous portions provides a pleasant warming effect by raising the temperature of the mixed portions by between 5-25° C. from room temperature.

Another aspect of the present invention provides that the zeolite is selected from the group consisting of naturally occurring and synthetic crystalline metal aluminosilicates. A further aspect of the present invention provides that the zeolite is sodium silicoaluminate. A further aspect of the present invention provides that the anhydrous portion comprises from 15-55 weight % of sodium aluminosilicate as the zeolite.

Yet another aspect of the present invention provides that the anhydrous portion carrier comprises one or more members selected from the group consisting of glycols, liquid polyethylene glycols, linear and branched esters having C₈-C₂₂ in each portion on either side of the ester linkage, linear and branched chain ethoxylates having 8-22 carbons, mineral oils, hydrogenated castor oil, and PEG hydrogenated castor oils and mixtures of two or more of the foregoing.

Another aspect of the present invention provides that the carrier of the anhydrous portion comprises at least one member selected from the group consisting of butylene glycol, PEG-400 NF, Glycereth-26, light mineral oil, octyl isononanoate, PEG-40 hydrogenated castor oil, and sodium behenoyl lactylate and mixtures of two or more of the foregoing.

Still another aspect of the present invention provides that the carrier of the anhydrous portion comprises at least one member selected from the group consisting of glycerol, propylene glycol, methylpropanediol, hexylene glycol, cocoglycerides, capric/caprylic triglyceride, lanolin oil, (C₂-C₂₀)isoparaffin, (C₁₂-C₁₅)alkyl benzoate, diisopropyl sebacate, octyl octanoate, octyldodecyl neopentanoate, hexyl laurate, isopropyl myristate, dicaprylyl carbonate, dibutyl adipate, soluble glycols and mixtures of two or more of the foregoing.

In another aspect of the present invention the anhydrous portion additionally comprises a surfactant selected from the group consisting of anionic, nonionic, cationic, and amphoteric surfactants and mixtures of two or more of the foregoing.

A further aspect of the present invention provides that the carrier comprises PEG-400 NF, butylene glycol, PEG-40 hydrogenated castor oil, sodium behenoyl lactylate, and hydroxypropyl cellulose.

Another aspect of the present invention provides that the carrier comprises 5-40 weight % PEG 400 NF; 15-50 weight % butylene glycol; 1-5 weight % PEG-40 hydrogenated castor oil; 0.1-5 weight % sodium behenoyl lactylate; and, 0.1-5 weight % hydroxypropyl cellulose.

Yet another aspect of the present invention provides that the carrier described above further comprises from 0.1-25 weight % of at least one member selected from the group consisting essentially of glycereth-26, a mineral oil, and octyl isononannoate. In another aspect, the carrier further comprises from 0.1-0.4 weight % methyl paraben; and, from 0.1-0.4 weight % propyl paraben. In still another aspect, the carrier further comprises from 1.0-4.0 weight % of palmitamidotrimonium chloride or C₂₀-C₄₀ Pareth-10.

Another aspect of the present invention provides that the carrier comprises from 30-50 weight % sodium aluminosilicate; from 7-14 weight % PEG-400 NF; from 20-30 weight % butylene glycol; from 2-4 weight % PEG-40 hydrogenated castor oil; from 0.5-2 weight % sodium behenoyl lactylate; from 0.1-5 weight % hydroxypropyl cellulose; from 1-10 weight % glycereth-26; from 1-10 weight % light mineral oil; from 5-15 weight % octyl isononannoate; from 0.1-0.3 weight % methyl paraben; and from 0.5-2 weight % propyl paraben. A further aspect of the present invention provides that this carrier further comprises from 1.0-4.0 weight % of palmitamidotrimonium chloride or C₂₀-C₄₀ Pareth-10.

Another aspect of the present invention provides that the hydrous portion comprises water, thickeners or stabilizers, moisturizers, emollients, emulsifiers and one or more members selected from the group of carriers; preservatives; pH adjusters; opacifying agents; feel improving agents; antioxidants and fragrances.

In another preferred embodiment of the present invention provides a dual warming and moisturizing composition which comprises a hydrous portion comprising a moisturizing composition having a water content of at least 10 weight %; and an anhydrous portion comprising a zeolite, a carrier and a suspending or a dispersing agent; wherein (i) the anhydrous portion and the hydrous portion are provided in a single container which keeps said portions separate until use (ii), the anhydrous portion and the hydrous portion each have viscosity in the range of 1,000-400,000 centipoise, (iii) the viscosities of each of the hydrous portion and the anhydrous portion are matched within a range of up to ±15% of each other, and (iv) the anhydrous portion and the hydrous portion provide warming and moisturizing effects without externally added water when said portions are mixed together.

In another aspect of the present invention the anhydrous portion and the hydrous portion each have viscosity in the range of 10,000-200,000 cps, particularly from 20,000-90,000 cps, more particularly from 40,000-60,000 cps.

A further aspect of the present invention provides that the anhydrous portion to the hydrous portion is in the range of from about 3:1 to about 1:3, particularly from about 2:1 to about 1:2, more particularly about 1:1.

A further aspect of the present invention provides a packaged product for dispensing a composition which comprises a package which is a dual chambered container, wherein the dual chambered container comprises a hydrous portion comprising a moisturizing composition in a first chamber of the dual chamber, the hydrous portion having a water content of at least 10 weight %; and an anhydrous portion in a second chamber of the dual chamber, the anhydrous portion comprising a zeolite, a carrier and a suspending or a dispersing agent; wherein (i) the anhydrous portion and the hydrous portion are provided in a single container which keeps said portions separate until use (ii) the concentration of zeolite in the anhydrous portion is matched to the water content of the hydrous portion to provide a pleasant warming effect upon use and (iii) the anhydrous portion and the hydrous portion are combined to provide warming and moisturizing effects without externally added water.

Another aspect of the present invention provides a packaged product for dispensing a composition which comprises a package which is a dual chambered container, wherein the dual chambered container comprises a hydrous portion comprising a moisturizing composition in a first chamber of the dual chamber, the hydrous portion having a water content of at least 10 weight %; and an anhydrous portion in a second chamber of the dual chamber, the anhydrous portion comprising a zeolite, a carrier and a suspending or a dispersing agent; wherein (i) the anhydrous portion and the hydrous portion are provided in a single container which keeps said portions separate until use (ii), the anhydrous portion and the hydrous portion each have a viscosity in the range of 1,000-400,000 cps, (iii) the viscosities of each of the hydrous portion and the anhydrous portion are matched within a range of up to ±15% of each other, and (iv) the anhydrous portion and the hydrous portion are combined to provide warming and moisturizing effects without externally added water.

A further aspect of the present invention provides that the dual chambered container comprises an inner plastic tube (1) and an outer laminate tube (2) that are independent of each other and interlocked at a top opening head (3) and a bottom head (4), wherein each container comprises a communication opening (5) for the mutual dispensing of products and wherein the anhydrous portion and hydrous portion are dispensed at a constant rate in about equal quantities.

Yet another aspect of the present invention provides that the dual chambered container comprises a first container (6) for receiving a first product, a second container (7) for receiving a second product, wherein the first and second containers are interlocked at a top head (8) and a bottom head (9), and wherein each container comprises a communication opening forming a dual communication opening (10) for the simultaneous dispensing of products, and a pump device (11) for dispensing the first and second products simultaneously though the dual communication opening (10).

A further aspect of the present invention provides a method of heating and moisturizing skin which comprises applying to the skin the above listed compositions.

### BRIEF DESCRIPTION OF THE DRAWINGS

The detailed description of the invention will be better understood when read in conjunction with the appended drawings. The appended drawings are only for the purposes of illustrating the invention. The invention is not limited to the precise arrangements and instrumentalities shown.

FIG. 1 illustrates an embodiment of a dual tube type of dual chambered container.

FIG. 2 illustrates an embodiment of a top opening head of a dual tube type of dual chambered container.

FIG. 3 illustrates an embodiment of a communication opening of a dual tube type of dual chambered container.

FIG. 4 illustrates the front view of an embodiment of a twin dual dispenser type of dual chambered container.

FIG. 5 illustrates the side view of an embodiment of a twin dual dispenser type of dual chambered container.

### DETAILED DESCRIPTION OF THE DRAWINGS

The product for dispensing a dual warming and moisturizing composition of the invention includes a dual chambered container which can be any chamber with two containers known in the art which is capable of dispensing two different liquids, gels, pastes, or creams.

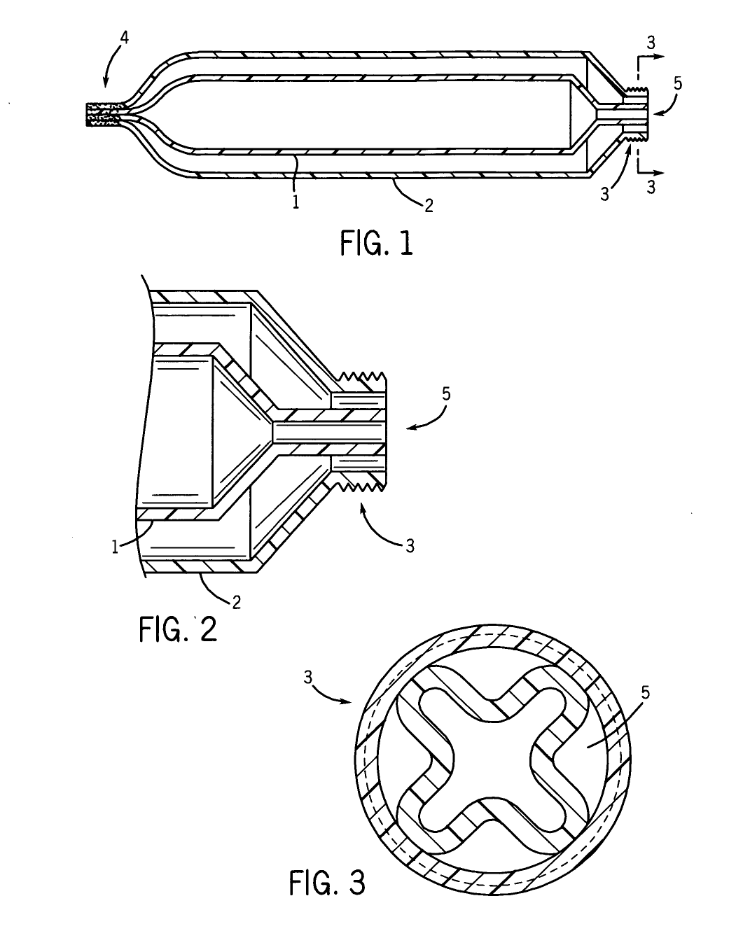

FIG. 1 shows a dual chambered container includes an inner plastic tube (1) and an outer laminate tube (2) that are independent of each other and interlocked at a top opening head (3) and a bottom head (4). Each dual chambered container comprises a communication opening (5) for the simultaneous dispensing of both the anhydrous and hydrous portions, particularly wherein the anhydrous portion and hydrous portion are distributed in approximately equal quantities and particularly at approximately equal rates. An example of this is the Dual-Tube® product (Cebal Tubes Europe, Paris, France). The Dual-Tube® product is a “tube in tube” packaging, and the outer laminated tube serves as an efficient external barrier for the protection of products. This Dual-Tube® product helps to ensure that formulations are kept separate until final application is made with approximately an even 50-50 ratio. In one embodiment of the present invention, the anhydrous portion and the hydrous portion in the Dual-Tube® product are mixed and allowed to react together to provide the finally combined mixture only at the time the portions are dispensed. Both portions are cleanly and evenly dispensed so that the user may rub and mix the portions together as they are applied to the skin.

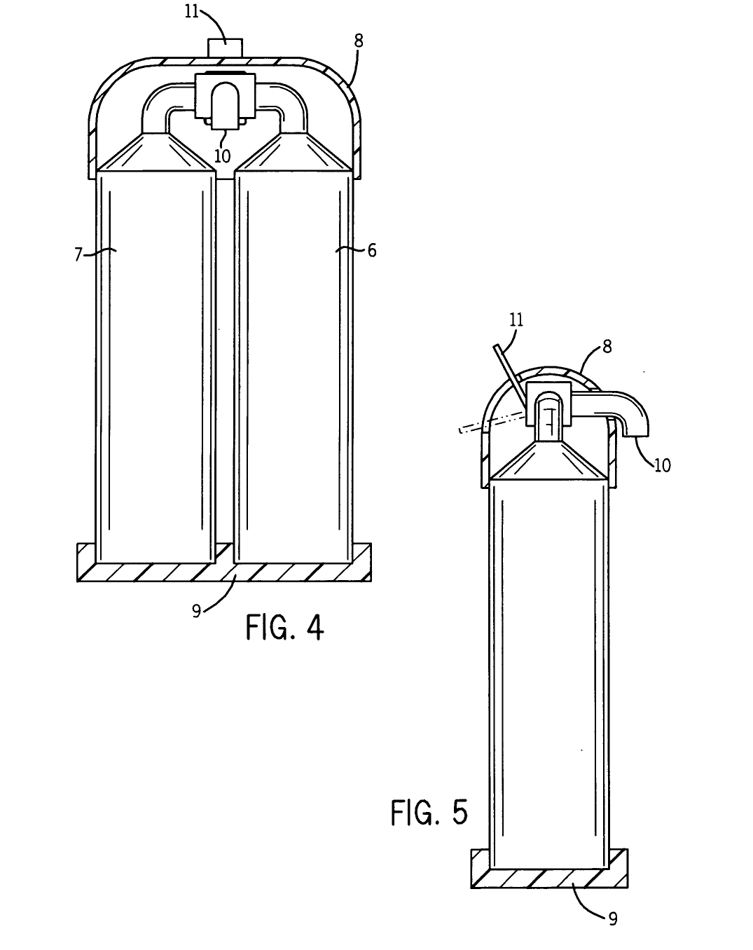

FIG. 4 shows a dual chambered container comprising a first container (6) for receiving a first product, and a second container (7) for receiving a second product. The first and second containers are each interlocked at a top head (8) and a bottom head (9). Each container comprises a communication opening forming a dual communication opening (10) for the simultaneous dispensing of products, and a pump device (11) for dispensing the first and second products simultaneously though the dual communication opening (10). An example of this dual chambered container is the Twin Dual Dispenser® product manufactured by WIKO (Exton, Pa.).

Other examples of dual containers that can be employed in the present invention include, but are not limited to, those disclosed in U.S. Pat. Nos. 5,862,949; 5,611,463; 5,967,372; 5,954,234; 6,286,520; 6,116,466; and 6,321,908, the contents of each being incorporated herein by reference as to their description of such containers.

### DETAILED DESCRIPTION OF THE INVENTION

The dual warming and moisturizing composition of the present invention is a product which provides a warming sensation while moisturizing the skin and comprises a hydrous portion and an anhydrous portion. The anhydrous portion comprises a zeolite and a carrier which are described herein. The hydrous portion comprises a lotion component, also referred to herein as lotion, which itself comprises a carrier and one or more ingredients selected from the group consisting of emollients, emulsifiers, humectants, and over-the-counter actives (for example sunscreens, skin protectants, topical analgesics and anti-itching agents).

The anhydrous portion and the hydrous portion are combined at the time of application to the skin to provide warming and moisturizing effects without the need for an external source of water. The concentration of zeolite in the anhydrous portion is matched to the water content of the hydrous portion to provide a pleasant warming effect upon use. A pleasant warming effect is found when, upon mixing the anhydrous and hydrous mixture, the temperature of the mixture is raised between 5-25° C. In any case, to provide a pleasant warming effect, the highest temperature achieved upon the mixing of the portions should not exceed 50° C. The matching of the concentration of zeolite in the anhydrous portion to the water content of the hydrous portion enables the dispensing of both portions at a constant rate, which also facilitates a similar heating experience when the product is used at different times. An example of an apparatus that dispenses two portions simultaneously in differing amounts, but at a constant rate is VERSADIAL® Dispensing System available from Versadial, New York, N.Y.

Additionally, the hydrous portion and the anhydrous portion may be formulated such that each has a viscosity in the range of 1,000-400,000 centipoise (“cps”), particularly in the range of 10,000-200,000 cps, more particularly in the range of 20,000-90,000 cps, and more particularly in the range from 40,000 cps to about 60,000 cps. The viscosities of each of the hydrous portion and the anhydrous portion are matched within a range of up to +15% of each other, particularly within a range of up to ±10% of each other, and, more particularly within a range of up to ±5% of each other. The matching of the viscosities of the hydrous and anhydrous portions enables the simultaneous dispensing of both portions at a constant rate and in similar amounts, which also facilitates a similar heating experience when the product is used at different times. Examples of an apparatus that dispenses two portions simultaneously at a constant rate and in similar amounts are discussed above.

Throughout this case the following terms, unless otherwise indicated, shall be understood to have the following meanings.

The term “anhydrous” means less than about 10 weight % of water, more particularly less than about 3 weight %, of water and, even more particularly, less than about 1 weight % of water for each portion.

The term “hydrous” means comprising water in an amount in the range of 10-95 water weight %, particularly 40-70 water weight %.

The term “composition” is intended to encompass a product comprising the specified ingredients in the specified amounts, as well as any product which results, directly or indirectly, from a combination of the specified ingredients in the specified amounts.

The term “fragrance” is intended to refer to a chemical or blend of chemicals that together have a desirable odor. Fragrances typically consist of a blend of chemicals, fragrant chemicals or fragrance materials. A large number of fragrance materials are known and used in various products such as perfumes, cosmetics, soaps, detergents, etc.

Throughout this specification, amounts are in weight % based on each component (hydrous or anhydrous) as referenced unless otherwise specified.

The zeolites of the anhydrous portion can be naturally occurring and/or synthetic crystalline metal aluminosilicates. The chemical composition, structure, preparation, and physical and chemical properties of such zeolites have been disclosed in numerous articles, patents and texts. These sources include D. W. Breck's book, Zeolite Molecular Sieves; Structure, Chemistry and Uses (Wiley-International 1974), which is incorporated herein by reference as to its description of such zeolites.

Synthetic zeolites are a particular group useful for the compositions of this invention. Non-limiting examples of synthetic zeolites include synthetic crystalline metal aluminosilicates such as those described in U.S. Pat. Nos. 2,882,243; 3,012,853; 3,130,007; and 3,329,621; which are all incorporated herein by reference as to their description of these zeolites.

Useful zeolites include, but are not limited to a zeolite known as Zeolite A. Zeolite A belongs to a class of zeolite with a selected crystal structure and is available from W.R Grace & Co., Columbia, Md. under the tradename Sylosiv®. An example of a useful Zeolite A is Sylosiv® A4.

Examples of other suitable synthetic zeolites include those prepared by combining aqueous solutions containing sources of silica, alumina, alumino-silicate gel which crystallizes upon hydro-thermal treatment to form an intermediate alumino-silicate gel which can be used in the anhydrous composition.

In a second embodiment, the intermediate alumino-silicate gel can be washed and dried to complete the preparation of the synthetic zeolite which can be used in the anhydrous composition.

In a third embodiment, the zeolite can be dehydrated by heating the zeolite at temperatures sufficient to substantially eliminate water, but below the decomposition point of the zeolite.

The zeolite is usually in the form of a powder and is suspended or dispersed in a carrier using a dispersing agent to create the anhydrous portion of the invention. A useful carrier or vehicle for the anhydrous portion must be liquid at room temperature and may itself be substantially anhydrous.

Care must be taken when choosing the zeolite and the concentration to be used. A pleasant warming effect is found when, upon mixing the anhydrous and hydrous mixture, the temperature change (ΔT) of the mixture results in a rise of between 5-25° C. This temperature change is dependant on both the concentration of the zeolite and the water content of the hydrous portion.

In Example 4, several concentrations of the synthetic zeolite sold under the brand name SYLOSIV® were mixed with varying amounts of water, and the temperature was immediately measured to determine the ΔT of the mixtures. All of the mixtures showed an increase in temperature after the addition of water, with the greatest change in temperature occurring with the addition of between 1.5-3.0 mL of water. The greatest temperature change was found with a 40% zeolite concentration (ΔT=21° C. at 2.5 mL of water added), whereas both the 27.5% or 30% zeolite concentrations exhibited a ΔT of 13-14° C. at 1.5-2.5 mL of water added. When water in excess of 2.5 mL was added, the ΔT decreased.

Therefore, one of ordinary skill in the art, using the teachings contained herein, can easily determine the appropriate concentration of the chosen zeolite to use when the water content of the hydrous portion is known. One would simply measure the temperature change of various concentrations of the chosen zeolite when added to the hydrous portion of choice. The optimal zeolite concentration is the concentration which yields the desired ΔT.

Examples of suitable carriers include glycols (particularly propylene glycol, dipropropylene glycol, butylene glycol, dibutylene glycol, hexylene glycol, glycerol pentylene glycol, ethoxydiglycol), liquid polyethylene glycols (particularly those having a molecular weight up to 400), linear and branched esters (particularly those having C₈-C₁₈ in each portion across the ester linkage “—C(O)O” with examples being sodium behenoyl lactylate, sodium stearoyl lactylate and octyl isononanoate), linear and branched chain ethoxylates (for example, having no more that 50 moles of ethoxylation), mineral oils (particularly light mineral oil), hydrogenated castor oil, and PEG hydrogenated castor oils (particularly PEG-40 hydrogenated castor oil).

A more particular group consists of one or more members selected from the group consisting of butylene glycol, PEG-400 NF, Glycereth-26, light mineral oil, octyl isononanoate, PEG-40 hydrogenated castor oil, sodium behenoyl lactylate. Another particular group consists of one or more members of the group consisting of Glycereth-26, light mineral oil, octyl isononannoate, and mixtures thereof. Yet another particular group consists of one or more members of the group consisting of PEG-40 hydrogenated castor oil, sodium behenoyl lactylate and mixtures thereof. It should be noted that the use of esters (especially octyl isononanoate) improves feel and reduces tackiness.

Another particular group of carriers comprises at least one member selected from the group consisting of butylene glycol, PEG-400 NF, Glycereth-26, light mineral oil, octyl isononanoate, PEG-40 hydrogenated castor oil, and sodium behenoyl lactylate.

Yet another particular group of carriers comprises at least one member selected from the group consisting of glycerol, propylene glycol, methylpropanediol, hexylene glycol, cocoglycerides, capric/caprylic triglyceride, lanolin oil, (C₁₂-C₂₀)isoparaffin, (C₁₂-C₁₅)alkyl benzoate, diisopropyl sebacate, octyl octanoate, octyldodecyl neopentanoate, hexyl laurate, isopropyl myristate, dicaprylyl carbonate, dibutyl adipate, soluble glycols and mixtures of two or more of the foregoing.

Non-limiting examples of substances that can be used as a substitute for or in combination with butylene glycol include glycerol, propylene glycol, methylpropanediol, hexylene glycol, and the like, or mixtures thereof.

Non-limiting examples of substances that can be used as a substitute for or in combination with PEG 400 NF include other higher molecular weight polyethylene glycols such as PEG 3350 or PEG 8000.

Non-limiting examples of substances that can be used as a substitute for or in combination with light mineral oil include petroleum, cocoglycerides, capric/caprylic triglyceride, lanolin oil, (C₁₂-C₂₀)isoparaffin, and the like, or mixtures of two or more of the foregoing.

Non-limiting examples of substances that can be used as a substitute for or in combination with octyl isononanoate include (C₁₂-C₁₅)alkyl benzoate, diisopropyl sebacate, octyl octanoate, octyldodecyl neopentanoate, hexyl laurate, isopropyl myristate, dicaprylyl carbonate, dibutyl adipate, and the like, or mixtures of two or more of the foregoing.

The anhydrous portion also requires the presence of a dispersing or suspending agent. The dispersing agent can be a surfactant including one or more members selected from the group consisting of anionic, nonionic, cationic or amphoteric surfactants and combinations thereof. Non-limiting examples include polyoxyethylene (hereinafter abbreviated as POE) hardened castor oil, POE alkyl ethers, POE branched alkyl ethers, POE fatty acid esters, POE glycerol fatty acid esters, POE sorbitan fatty acid esters, POE sorbitol fatty acid esters, POE hardened castor oil alkyl sulfates, POE alkyl sulfates, alkali metal salts of fatty acids, sorbitan fatty acid esters, glycerol fatty acid esters, alkyl polyglucosides, polyethylene glycol fatty acid esters, ether-modified silicones and combinations thereof. Suitable POEs include those having between 8-22 carbon atoms and the degree of ethoxylation (defined as moles ethylene oxide per molecule) is in the range from about 5 to about 150, particularly about 10 to about 10 and most particularly from about 10 to about 50.

Examples of suitable thickeners (also called suspending agents or viscosity modifiers) include cellulosic derivatives such as carboxymethyl cellulose, hydroxypropyl cellulose, hydroxypropylmethyl cellulose, solid polyethylene glycols such as PEG 3350, wax esters of behenic acid, such as stearyl behenate, lauryl behenate, cross-linked polyacrylic acids, polyacrylamides, carbomers, pluronics, celluloses, xanthan gums, guar gums, alginates, pectins, carrageenans, polyethylene glycol, polyvinyl alcohols, polyvinyl pyrrolidone, and starches.

Other optional ingredients may also be included, for example, one or more members selected from the group consisting of colors, fragrances, preservatives (e.g. diazolidinyl urea, butylated hydroxytoluene, iodopropynyl butylcarbamate, and parabens such as methyl paraben and propyl paraben), and agents to improve feel (e.g. cyclomethicone, and cationic surfactants such as palmitamidotrimonium chloride and distearyl dimonium chloride).

When parabens are used as preservatives in the anhydrous portion of the compositions of the invention they may be used in amounts of 0.05-0.4% by weight, particularly 0.1-0.3% and, more particularly, about 0.2% by weight of methyl paraben; or 0.05-0.4% by weight, particularly 0.1-0.2% and, more particularly, about 0.1% of propyl paraben.

The anhydrous portion can be formulated to have a viscosity ranging from about 1,000 cps to about 400,000 cps, or from about 10,000 cps to about 200,000 cps, of from about 20,000 cps to about 90,000 cps, or from about 40,000 cps to about 60,000 cps.

In a first particular embodiment, the anhydrous portion comprises:

(a) 15-55 weight % (particularly 30-50%) of a zeolite; and

(b) 45-85 weight % of a carrier comprising a member selected from the group consisting of glycerin, PEG having a molecular weight in the range of 200-2000 Daltons, butylene glycol, and mixtures of two or more of the foregoing; and

(c) 0.1-10 weight % of a suspending agent selected from the group consisting of cellulose derivatives based on the total weight of the anhydrous portion.

In a second particular embodiment, the anhydrous portion comprises 5%-40% by weight, particularly 7%-14% by weight and, more particularly, about 10% of a polyethylene glycol such as PEG 400 NF.

In a third particular embodiment, the anhydrous portion comprises 15%-50% by weight, particularly 20%-30% and, more particularly, about 25% of butylene glycol.

In a fourth particular embodiment, the anhydrous portion comprises 1%-5% by weight, particularly 2-4% and, more particularly, about 3% of PEG-40 hydrogenated castor oil.

In a fifth particular embodiment, the anhydrous portion comprises 0.1%-5% by weight, particularly 0.5-2% and, more particularly, about 3% of PEG-40 hydrogenated castor oil.

In a sixth particular embodiment, the anhydrous portion comprises 0.1%-5% by weight, particularly 0.5-2% and, more particularly, about 1% of sodium behenoyl lactylate.

In a seventh particular embodiment, the anhydrous portion comprises at least one of:

(a) 0.1-25% by weight, particularly 1-10% and, more particularly, about 5% of glycereth-26;

(b) 0.1-25% by weight, particularly 1-10% and, more particularly, about 5% of light mineral oil;

(c) 0.1-25% by weight, particularly 5-15% and, more particularly, about 8% of octyl isononannoate; ranging from about 0% to about 25% by weight, or from about 5% to about 15% by weight, or about 8% by weight;

In an eighth particular embodiment, the anhydrous portion comprises any of the previous 7 embodiments in combination with 0.1%-5% by weight, particularly 0.5-2% and, more particularly, about 1% of hydroxypropyl methyl cellulose.

In a ninth particular embodiment, the anhydrous portion comprises a zeolite, PEG-400 NF, butylene glycol, PEG-40 hydrogenated castor oil, sodium behenoyl lactylate, and hydroxypropyl cellulose.

The hydrous portion can be any moisturizing liquid, cream, gel or lotion, and mixtures of the foregoing provided the viscosity limitations are met and are matched to the anhydrous portion as previously described. The lotion can be either oil in water (o/w), water-in-oil (w/o) emulsion, or an aqueous gel composition.

Non-limiting examples of hydrous portions are those comprising water according to the ranges described above, and one or more members selected from the group consisting of:

(a) thickeners or stabilizers (e.g. cetyl alcohol, xanthan gums, cross-linked polyacrylic acids, polyacrylamides, carbomers, pluronics, celluloses, guar gums, alginates, pectins, carrageenans, polyethylene glycol, polyvinyl alcohols, polyvinyl pyrrolidone, and starches);

(b) moisturizers (e.g. polyethylene, polypropylene, and sodium styrene-based copolymers, glycerin, water-soluble such as sorbitol, hydrolyzed proteins, urea, hydrolyzed starch, hydroxy acids such as lactic acid and fruit acids and salt derivatives thereof, pyrrolidone carboxylic acid, aloe vera gel, cucumber juice, mineral oils, squalene, and tocophenol)

(c) emollients (e.g. propylene glycol dicaprylate/dicaprate, isopropyl isostearate, tri (PPG-3 myristyl ether) citrate, fatty alkoxylate esters of aliphatic or aromatic, dicarboxylic or tricarboxylic acids);

(d) emulsifiers (e.g. PEG-40 stearate, emulsifying wax, lecithin, hydrogenated castor oil, PEG hydrogenated castor oils);

(e) carriers (e.g. glycols, liquid polyethylene glycols, linear and branched chain ethoxylates);

(f) preservatives (e.g. diazolidinyl urea, butylated hydroxytoluene, iodopropynyl butylcarbamate);

(g) pH adjusters (e.g. sodium hydroxide, sodium benzoate); chelating agents (e.g. disodium EDTA);

(h) opacifying agents (e.g. titanium dioxide)

(i) agents to improve feel (e.g. cyclomethicone, cationic surfactants),

(j) antioxidants (e.g. tocopheryl acetate),

(k) anti-wrinkle agents (e.g. retinyl palmitate) and

(l) fragrances.

Hydrous moisturizing compositions are well known in the art. Examples of commercially available moisturizing compositions which may be utilized in the current invention include LUBRIDERM® Advanced Therapy Lotion, LUBRIDERM® Seriously Sensitive, LUBRIDERM® Skin Nourishing, LUBRIDERM® Daily Moisture Lotion, LUBRIDERM® UV Lotion, LUBRIDERM® with Oat extract, LUBRIDERM® Sea Kelp Extract, LUBRIDERM® with Shea & Cocoa Butter, VASELINE® Intensive Care®, JERGENS® lotions, AVENO® lotions and creamy moisturizing oil, NEUTROGENA® Visibly Firm™ body lotion, SUAVE® lotions, CUREL® lotions, KERI® lotions. Of course, it may be necessary to modify the moisturizing composition to meet the viscosity limitations and to match the viscosity to the anhydrous portion as previously described.

In a first particular embodiment, the hydrous portion comprises water, mineral oil, petrolatum, sorbitol solution, stearic acid, lanolin, lanolin alcohol, cetyl alcohol, glyceryl stearate/PEG-100 stearate, triethanolamine, dimethicone, propylene glycol, microcrystalline wax, Tri (PPG-3 myristyl ether) citrate, disodium EDTA, methylparaben, ethylparaben, propylparaben, fragrance, xanthan gum, butylparaben, and methyldibromo glutaronitrile.

In a second particular embodiment, the hydrous portion comprises water, butylene glycol, mineral oil, petrolatum, glycerin, cetyl alcohol, propylene glycol dicaprylate/dicaprate, PEG-40 stearate, (C₁₁-C₁₃)isoparaffin, glyceryl stearate, tri (PPG-3 myristyl ether) citrate, emulsifying wax, dimethicone, DMDM hydantoin, methylparaben, carbomer 940, ethylparaben, propylparaben, titanium dioxide, disodium EDTA, sodium hydroxide, butylparaben, and xanthan gum.

In a third particular embodiment, the hydrous portion comprises octyl methoxycinnamate, octyl salicylate, oxybenzone, purified water, (C₁₂-C₁₅)alkyl benzoate, cetearyl alcohol (and) ceteareth-20, cetyl alcohol, glyceryl monostearate, propylene glycol, petrolatum, diazolidinyl urea, triethanolamine, diosodium EDTA, xanthan gum, acrylates/(C₁₀-C₃₀)alkyl acrylate crosspolymer, tocopheryl acetate, iodopropynyl butylcarbamate, carbomer, and fragrance.

In a fourth particular embodiment, the hydrous portion comprises octinoxate, octisalate, oxybenzone, purified water, C₁₂-C₁₅ alkyl benzoate, cetearyl alcohol (and) ceteareth-20, cetyl alcohol, glyceryl monostearate, propylene glycol, white petrolatum, diazolidinyl urea, trolamine, edetate disodium, xanthan gum, acrylates/C₁₀-C₃₀ alkyl acrylate crosspolymer, vitamin E, iodopropynyl butylcarbamate, fragrance, and carbomer.

In a fifth particular embodiment, the hydrous portion comprises water, butylene glycol, mineral oil, petrolatum, glycerin, cetyl alcohol, propylene glycol dicaprylate/dicaprate, PEG-40 stearate, C₁₁-C₁₃ isoparaffin, glyceryl stearate, Tri (PPG-3 myristyl ether) citrate, emulsifying wax, dimethicone, DMDM hydantoin, methylparaben, carbomer, ethylparaben, propylparaben, titanium dioxide, disodium EDTA, sodium hydroxide, butylparaben, and xanthan gum.

In a sixth particular embodiment, the hydrous portion comprises water, glycerin, petrolatum, caprylic/capric triglycerides, glyceryl stearate SE, cetyl alcohol, cocoa butter, castor oil, cetearyl alcohol, polysorbate 60, oat kernel extract, glyceryl stearate, PEG-100 stearate, diazolidinyl urea, xanthan gum, disodium EDTA, fragrance, and iodopropynyl butylcarbamate.

In a seventh particular embodiment, the hydrous portion comprises water, glycerin, glyceryl sterate SE, cetyl alcohol, emulsifying wax, petrolatum, caprylic/capric triglyceride, castor oil, octyldodecanol, dimethicone, diazolidinyl urea, propylene glycol, xanthan gum, disodium EDTA, fragrance, giant kelp leaf extract, and iodopropynyl butylcarbamate.

In a eighth particular embodiment, the hydrous portion comprises water, glycerin, cetyl alcohol, glyceryl sterate SE, petrolatum, emulsifying wax, caprylic/capric triglyceride, castor oil, octyldodecanol, shea butter, cocoa butter, dimethicone, tocopheryl acetate, diazolidinyl urea, xanthan gum, disodium EDTA, fragrance, and iodopropynyl butylcarbamate.

In a ninth particular embodiment, the hydrous portion comprises water, ethyl alcohol, glycerin, isopropyl myristate, propylene glycol, tocopheryl acetate, aminomethyl propanol, carbomer, and a fragrance.

In a tenth particular embodiment, the hydrous portion comprises water, glycerin, carbomer 940, stearic acid, glyceryl stearate, cetyl alcohol, isopropyl palmitate, cetyl phosphate, dl-menthol, methyl salicyclate and triethanolamine.

The ratio of the anhydrous portion to the hydrous portion can be from about 3:1 to about 1:3, particularly from about 2:1 to about 1:2 and, more particularly, about 1:1.

The invention contemplates any combination of the previously described anhydrous and hydrous portions, provided, of course, that the viscosity limitations are met and are matched between the anhydrous and the hydrous portions as previously described. Furthermore, the importance of the compatibility and stability of the hydrous will be understood by those of skill in the art. Those hydrous compositions which would be appropriate for use in the present invention are those which do not settle out into constituent components over a reasonable period of time, generally, at least one year.

As noted above, the viscosities of the hydrous and anhydrous portions should be matched within the tolerances described above. While viscosities may be measured in a variety of ways, the test described below in Example 4 should be used for the purpose of this invention.

The anhydrous composition is added to one compartment of a suitable dual chamber dispenser and the moisturizing lotion containing water is added to the other compartment of the dual chamber dispenser. The dual chamber dispenser then dispenses each component at about the same time, particularly in about a 1:1 ratio.

The method of heating and moisturizing skin using the composition of the invention includes applying both the anhydrous portion and the hydrous portion to the skin from a dual chambered container, wherein the dual chambered container comprises a first chamber containing the anhydrous composition, and a second chamber containing the hydrous composition, and the hands are rubbed together to combine the hydrous and anhydrous portions to release the warming sensation as the skin is moisturized.

### EXAMPLES

The following Examples are offered as illustrative of the invention and are not to be construed as limitations thereon. In the Examples and elsewhere in the description of the invention, chemical symbols and terminology have their usual and customary meanings. Comprising shall be read as including the subgroups of consisting and consisting essentially of. In the Examples as elsewhere in this application, values for formulas, molecular weights and degree of ethoxylation or propoxylation are averages. Temperatures are in degrees C. unless otherwise indicated. The amounts of the components are in weight percents based on the standard described; if no other standard is described then the amounts are in weight percents based on the total weight of the composition. It will be understood that numerous additional formulations can be prepared without departing from the spirit and scope of the present invention.

### Example 1

### Anhydrous Composition

Preparation of anhydrous composition may be made in amounts of about 200-1000 grams (typically 500 g) with the following proportions of ingredients:

| Phase | Ingredient | weight % |
|---|---|---|
| A | Butylene Glycol | 24.7 |
| A | PEG 400 NF | 10.0 |
| A | Glycereth-26 | 5.0 |
| A | Mineral Oil | 5.0 |
| A | Octyl Isononanoate | 8.0 |
| A | PEG-40 Hydrogenated Castor Oil | 3.0 |
| A | Sodium Behenoyl Lactylate | 1.0 |
| A | Palmitamidotrimonium chloride | 2.0 |
| A | Methyl paraben | 0.2 |
| A | Propyl paraben | 0.1 |
| B | Hydroxypropyl cellulose | 1.0 |
| C | Sodium silicoaluminate (zeolite) | 40.0 |

All the phase A ingredients are added to the beaker. The zeolite employed is sodium silicoaluminate (Sylosiv® A4 supplied by W.R. Grace & Co.). The mixture is heated to 80-85° C. and mixed for about 15 minutes with an overhead mixer at a speed of about 500 rpm. The heat is turned off and the phase B ingredient is slowly added to the solution as it is cooling to about 60° C. with increased mixing at a speed of about 1000 rpm. The solution is mixed and allowed to cool until it reaches a temperature of about 40-45° C. and the B ingredient is completely solubilized as evidenced by a clear appearance to the mixture. The phase C ingredient is then added slowly to the solution with mixing at 1000 rpm for about 10-15 minutes.

### Examples 2-3

### Anhydrous Compositions

### Example 2

The method of Example 1 may be repeated with the types and amounts of ingredients listed below.

| Phase | Ingredient | weight % |
|---|---|---|
| A | Butylene Glycol | 34.1 |
| A | PEG 400 NF | 10.0 |
| A | Glycereth-26 | 5.0 |
| A | Light Mineral Oil | 5.0 |
| A | Octyl Isononanoate | 8.0 |
| A | PEG-40 Hydrogenated Castor Oil | 3.0 |
| A | Sodium Behenoyl Lactylate | 1.6 |
| A | C₂₀–C₄₀ Pareth-10 | 2.0 |
| A | Methyl paraben | 0.2 |
| A | Propyl paraben | 0.1 |
| B | Hydroxypropyl cellulose | 1.0 |
| C | Sodium silicoaluminate (zeolite) | 30.0 |

### Example 3

The method of Example 1 may be repeated with the types and amounts of ingredients listed below.

| Phase | Ingredient | weight % |
|---|---|---|
| A | Butylene Glycol | 24.7 |
| A | PEG 400 NF | 10.0 |
| A | Glycereth-26 | 5.0 |
| A | Light Mineral Oil | 5.0 |
| A | Octyl Isononanoate | 8.0 |
| A | PEG-40 Hydrogenated Castor Oil | 3.0 |
| A | Sodium Behenoyl Lactylate | 1.0 |
| A | C₂₀–C₄₀ Pareth-10 | 2.0 |
| A | Methyl paraben | 0.2 |
| A | Propyl paraben | 0.1 |
| B | Hydroxypropyl cellulose | 1.0 |
| C | Sodium silicoaluminate (zeolite) | 40.0 |

### Example 4

### SYLOSIV® Adsorption Heat Test

Preparations of formulations containing varying concentrations of SYLOSIV® were made to test the heat of adsorption upon addition of different amounts of water. SYLOSIV® was diluted in propilenglicol p.a. to the desired concentration, and the baseline temperature was taken for each formulation. To the formulations, varying amounts of water was added using a magnetic stirrer to achieve even distribution, and the temperature was then taken within 15 seconds of the addition of water. The temperature was also taken over time for a control sample which did not have any water added. The control confirmed that the temperature change was due to the addition of water, and not the influence of the magnetic stirrer.

| | [SYLOSIV ®] | [SYLOSIV ®] | |
|---|---|---|---|
| mL of water | 28.5% | 30% | [SYLOSIV ®] 40% |
| added | Temperature ° C. | Temperature ° C. | Temperature ° C. |
| 0 | 24.5 | 23 | 24 |
| 0.5 | 30 | 28 | 30 |
| 1 | 36 | 34 | 35 |
| 1.5 | 38 | 37 | 40 |
| 2 | 38 | 37 | 44 |
| 2.5 | 37 | 37 | 45 |
| 3 | — | — | 45 |
| 4.5 | 37 | 36 | — |
| 5 | — | — | 44 |

Control:

| Time (sec) | Temperature ° C. |
|---|---|
| 0 | 24 |
| 30 | 24 |
| 60 | 24 |
| 90 | 24 |
| 120 | 24 |

### Example 5

### Viscosity Measurements for Anhydrous Compositions

Viscosity measurements were performed on the compositions listed for Examples 1-3 using a Brookfield Viscometer RV spindle #6 and speed=10 rpm at room temperature. Readings were taken for initial viscosity and also after 1 minute and were recorded as listed in Table B. This method may also be used for any viscosity measurements described in this application.

TABLE B

| Product | Initial Viscosity (cP) | Viscosity 1 minute (cP) |
|---|---|---|
| Anhydrous Composition | 50,000 | 43,000 |
| Ex. 1 | | |
| Anhydrous Composition | 40,000 | 24,400 |
| Ex. 2 | | |
| Anhydrous Composition | 88,000 | 78,000 |
| Ex. 3 | | |

### Example 5

### Moisturizing Compositions

Preparation of the complete dual warming and moisturizing composition may be made as follows. Equal amounts of the anhydrous composition from Examples 1-3 can be loaded into a first compartment of a dual chamber container, for example as described for FIGS. 1 and 2. A hydrous portion as described above for the embodiments useful in this invention or a typical moisturizing lotion (for example, products sold under the following names: LUBRIDERM® Advanced Therapy Lotion, LUBRIDERM® Seriously Sensitive, LUBRIDERM® Skin Nourishing, LUBRIDERM® Daily Moisture Lotion, LUBRIDERM® UV Lotion, LUBRIDERM® with Oat extract, LUBRIDERM® Sea Kelp Extract, LUBRIDERM® with Shea & Cocoa Butter, VASELINE® Intensive Care®, JERGENS® lotions, AVENO® lotions and creamy moisturizing oil, NEUTROGENA® Visibly Firm™ body lotion, SUAVE® lotions, CUREL® lotions, KERI® lotions, or mixtures thereof having a water content and viscosity values according to the limits described above, wherein the viscosities of the anhydrous portion and the hydrous portion are matched as described above are loaded into a second compartment of the container.

### Example 6

### External Analgesic Compositions

Preparation of the complete dual warming and analgesic composition may be made as follows. Equal amounts of the anhydrous composition from Examples 1-3 can be loaded into a first compartment of a dual chamber container, for example as described for

FIGS. 1 and 2. A hydrous portion as described above or a typical counterirritant/analgesic lotion (for example, products sold under the following names: BEN-GAY® Original Gel, BEN-GAY® Vanishing Scent Gel, BEN-GAY® Ultra Strength Gel, BEN-GAY® Greaseless Pain Relieving Cream, BEN-GAY® ICE Extra Strength or BEN-GAY® Arthritis Extra Strength having a water content and viscosity values according to the limits described above, wherein the viscosities of the anhydrous portion and the hydrous portion are matched as described above are loaded into a second compartment of the container.

### Example 7

### Skin Sanitizing Compositions

Preparation of the complete dual warming and sanitizing composition may be made as follows. Equal amounts of the anhydrous composition from Examples 1-3 can be loaded into a first compartment of a dual chamber container, for example as described for FIGS. 1 and 2. A hydrous portion as described above or a typical skin sanitizing lotion (for example, products sold under the following names: PURELL® Instant Hand Sanitizer, PURELL® Instant Hand Sanitizer Moisture Therapy, PURELL® Instant Hand Sanitizer Ocean Mist, PURELL® Instant Hand Sanitizer Spring Bloom or PURELL® with Aloe Instant Hand Sanitizer having a water content and viscosity values according to the limits described above, wherein the viscosities of the anhydrous portion and the hydrous portion are matched as described above are loaded into a second compartment of the container.

It is to be understood that many modifications and variations may be devised given the above description of the principles of the invention. It is intended that all such modifications and variations can be considered as within the spirit and scope of this invention, as it is defined in the following claims.

What is claimed is:

1. A dual warming and moisturizing composition comprising:
a) a hydrous portion comprising a moisturizing composition having a water content of at least 10 weight %; and
b) an anhydrous portion comprising a zeolite, a carrier and a suspending or a dispersing agent;
wherein (i) the anhydrous portion and the hydrous portion are provided in a single container which keeps said portions separate until use, (ii) the concentration of zeolite in the anhydrous portion is matched to the water content of the hydrous portion to provide a pleasant warming effect upon use and (iii) the anhydrous portion and the hydrous portion provide warming and moisturizing effects without externally added water when said portions are mixed together.

2. The composition according to claim 1, wherein upon mixing the anhydrous and hydrous mixture, the temperature of the mixture rises from about 5° C. to about 25° C.

3. The composition according to claim 1, wherein the zeolite is sodium silicoaluminate.

4. The composition according to claim 1, wherein the anhydrous portion comprises from 15-55 weight % of sodium aluminosilicate.

5. The composition according to claim 1, wherein the carrier comprises one or more members selected from the group consisting of glycols; liquid polyethylene glycols; linear and branched esters having a C₈-C₂₂ moiety in each portion on either side of the ester linkage; linear and branched chain ethoxylates having 8-22 carbons; mineral oils; hydrogenated castor oil; PEG hydrogenated castor oils; and mixtures of two or more of the foregoing.

6. The composition according to claim 5 wherein the carrier comprises at least one member selected from the group consisting of butylene glycol; PEG-400 NF; Glycereth-26; light mineral oil; octyl isononanoate; PEG-40 hydrogenated castor oil; sodium behenoyl lactylate; glycerol; propylene glycol; methylpropanediol; hexylene glycol; cocoglycerides; capric/caprylic triglyceride; lanolin oil; (C₁₂-C₂₀)isoparaffin; (C₁₂-C₁₅)alkyl benzoate; diisopropyl sebacate; octyl octanoate; octyldodecyl neopentanoate; hexyl laurate; isopropyl myristate; dicaprylyl carbonate; dibutyl adipate; soluble glycols; and mixtures of two or more of the foregoing.

7. The composition according to claim 1 wherein the anhydrous portion additionally comprises a dispersing agent selected from the group consisting of anionic, nonionic, cationic, and amphoteric surfactants and mixtures of two or more of the foregoing.

8. The composition according to claim 1 wherein the anhydrous portion additionally comprises a suspending agent selected from the group consisting of cellulosic derivatives; solid polyethylene glycols; wax esters of behenic acid; cross-linked polyacrylic acids; polyacrylamides; carbomers; pluronics; celluloses; xanthan gums; guar gums; alginates; pectins; carrageenans; polyethylene glycol; polyvinyl alcohols; polyvinyl pyrrolidone; starches; and mixtures of two or more of the foregoing.

9. The composition according to claim 5, wherein the carrier comprises PEG-400 NF, butylene glycol, PEG-40 hydrogenated castor oil, sodium behenoyl lactylate, and hydroxypropyl cellulose.

10. The composition according to claim 5 comprising:
5-40 weight % PEG 400 NF;
15-50 weight % butylene glycol;
1-5 weight % PEG-40 hydrogenated castor oil;
0.1-5 weight % sodium behenoyl lactylate;
0.1-5 weight % hydroxypropyl cellulose;
0.1-25 weight % glycereth-26,
0.1-25 weight % a mineral oil;
0.1-25 weight % octyl isononannoate;
0.05-0.4 weight % methyl paraben; and,
0.05-0.4 weight % propyl paraben.

11. The composition according to claim 13 further comprising from 1.0-4.0 weight % palmitamidotrimonium chloride and from 1.0-4.0 weight % C₂₀-C₄₀ Pareth-10.

12. The composition according to claim 5 comprising
30-50 weight % sodium aluminosilicate;
7-14 weight % PEG-400 NF;
20-30 weight % butylene glycol;
2-4 weight % PEG-40 hydrogenated castor oil;
0.5-2 weight % sodium behenoyl lactylate;
0.1-5 weight % hydroxypropyl cellulose;
1-10 weight % glycereth-26;
1-10 weight % light mineral oil;
5-15 weight % octyl isononannoate;
0.1-0.3 weight % methyl paraben; and
0.5-2 weight % propyl paraben.

13. The composition according to claim 1, wherein the hydrous portion comprises water; thickeners or stabilizers; moisturizers; emollients; emulsifiers; and one or more members selected from the group of carriers; preservatives; pH adjusters; opacifying agents; feel improving agents; antioxidants and fragrances.

14. The composition according to claim 1, wherein the ratio of the anhydrous portion to the hydrous portion is in the range of from about 3:1 to about 1:3.

15. The composition according to claim 1, wherein the ratio of the anhydrous portion to the hydrous portion is about 1:1.

16. A dual warming and moisturizing composition comprising:
a) a hydrous portion comprising a moisturizing composition having a water content of at least 10 weight %; and
b) an anhydrous portion comprising a zeolite, a carrier and a suspending or a dispersing agent;
wherein (i) the anhydrous portion and the hydrous portion are provided in a single container which keeps said portions separate until use, (ii) the anhydrous portion and the hydrous portion each have a viscosity in the range of 1,000-400,000 centipoise, (iii) the viscosities of each of the hydrous portion and the anhydrous portion are matched within a range of up to +15% of each other, and (iv) the anhydrous portion and the hydrous portion provide warming and moisturizing effects without externally added water when said portions are mixed together.

17. The composition according to claim 16, wherein the anhydrous portion and the hydrous portion each have viscosity in the range of 20,000-90,000 centipoise.

18. A packaged product for dispensing a composition comprising a package which is a dual chambered container, wherein the dual chambered container comprises:
a) a hydrous portion comprising a moisturizing composition in a first chamber of the dual chamber, the hydrous portion having a water content of at least 10 weight %; and
b) an anhydrous portion in a second chamber of the dual chamber, the anhydrous portion comprising a zeolite, a carrier and a suspending or a dispersing agent;
wherein (i) the anhydrous portion and the hydrous portion are provided in a single container which keeps said portions separate until use, (ii) the concentration of zeolite in the anhydrous portion is matched to the water content of the hydrous portion to provide a pleasant warming effect upon use and (iii) the anhydrous portion and the hydrous portion provide warming and moisturizing effects without externally added water when said portions are mixed together.

19. The product according to claim 18, wherein the dual chambered container comprises an inner plastic tube and an outer laminate tube that are independent of each other and interlocked at a top opening head and a bottom head, wherein each container comprises a communication opening for the mutual dispensing of products and wherein the anhydrous portion and hydrous portion are dispensed at a constant rate in about equal quantities.

20. The product according to claim 18, wherein the dual chambered container comprises a first container for receiving a first product, a second container for receiving a second product, wherein the first and second containers are interlocked at a top head and a bottom head, and wherein each container comprises a communication opening forming a dual communication opening for the simultaneous dispensing of products, and a pump device for dispensing the first and second products simultaneously though the dual communication opening.
